(19) United States
(12) Patent Application Publication
(10) Pub. No.: US 2023/0190594 A1
(43) Pub. Date: Jun. 22, 2023

(54) COMPOSITIONS COMPRISING PERSONAL CARE PRODUCTS AND RELATED METHODS

(71) Applicant: ONE FUN COMPANY, INC., Southborough, MA (US)

(72) Inventors: Yuhan LEE, Cambridge, MA (US); Allison Quinn DIVINCENZO, Westport, CT (US); Jeffrey Michael KARP, Chestnut Hill, MA (US); Irena BARBULOVIC-NAD, Toronto (CA); Bryce William CUSHING, Framingham, MA (US)

(21) Appl. No.: 17/927,630

(22) PCT Filed: May 26, 2021
(86) PCT No.: PCT/US2021/034216

§ 371 Date: Nov. 23, 2022

Related U.S. Application Data
(60) Provisional application No. 63/030,250, filed on May 26, 2020.

(51) Int. Cl. A61K 8/11; A61K 8/73; A61Q 19/10

(52) U.S. Cl. CPC A61K 8/11; A61K 8/733; A61Q 19/10

(57) ABSTRACT

Compositions comprising personal care products are described. The composition may include a gel-like material and one or more personal care products enclosed within and/or integrated into the gel-like material. The composition may be used to deliver a personal care product by applying compression force to the composition to release the personal care product from the gel-like material.

### CROSS REFERENCE TO RELATED APPLICATIONS

This application claims priority to U.S. Provisional Application No. 63/030,250, filed on May 26, 2020, which is incorporated by reference herein in its entirety.

### TECHNICAL FIELD

The present disclosure generally relates to compositions comprising personal care products. For example, the present disclosure includes capsules comprising a flexible gel-like material and one or more personal care products at least partially integrated into the gel-like material and methods of use thereof. The capsules herein may deliver a personal care product by releasing it from the gel-like material as the capsule deforms and bursts under compression force and/or upon activation with water or an aqueous solution.

### BACKGROUND

Personal care products including personal hygiene products, cosmetics and wellness products come in a variety of forms including powder, liquid, solid bars, and lately liquid and powder pods. Pods are small packages for single use containing several milliliters of personal care product in a liquid or powder form. This form is suitable for use in personal care travel kits. Most pods are made of water soluble, thin films encapsulating a personal care product, e.g., shampoo, conditioner, and shaving cream. Water soluble films dissolve when in contact with water and in that way, the pods release the personal care product content.

### SUMMARY

The present disclosure provides compositions and methods for single use packaging of personal care products. Product delivery and release may be achieved by applying compression force to the composition and/or the packaging of the composition.

According to some examples herein, the present disclosure provides embodiments of a flexible, capsule made of compressible gel-like material and containing one or more personal care products, and embodiments of a method of delivering the contained personal care products. Personal care products suitable for the compositions and methods herein include, but are not limited to, personal hygiene products (e.g., hand soap, body wash, shampoo, conditioner, bubble bath, bath bombs, cleanser, body and facial scrub, toothpaste, mouthwash, shaving cream, hair removal formulations, personal lubricant, hand sanitizer, anti-bacterial soap, disinfectant, deodorant, face wash, combination shampoo-conditioner-body wash, hair gel, mousse, nail polish remover, wax, pomade etc.), cosmetic products (e.g., moisturizer, detangler, cream, hand and body lotion, sunscreen lotion, lip balm, makeup, toner, additives, glitter, active ingredients, body paint, lip gloss, nail polish, hand cream etc.), and wellness products (e.g., vitamins, supplements, eye drops etc.).

The flexible capsule may comprise one or more gel-like materials. The capsule may be in the form of a sphere, heart, star, cube or any other shape with outer dimensions of several millimeters to several centimeters. In some examples, the capsule may consist of or consist essentially of one or more gel-like materials integrating a personal care product. In some examples, the capsule comprises a membrane and a core, wherein the membrane comprises one or more gel-like materials encapsulating a personal care product in the core. The gel-like material(s) may comprise water soluble, water semi-soluble, water insoluble components or any combination thereof, including but not limited to, locust bean gum polysaccharide, softening silicone, alginate, agarose, agar, pectin, xanthan, gelatin, hydroxy propyl methyl cellulose (HPMC), carrageenan, arrowroot, cellulose and cellulose derivatives, hyaluronan, elastin, chitosan, poly(vinyl alcohol), poly(acrylic acid), poly(ethylene glycol), poly(vinyl acetate), and combinations thereof. To form the gel-like material, the water soluble, water semi-soluble, water insoluble components or any combination of aforementioned may be crosslinked via a chemical interaction including, but not limited to, covalent bonds, electrostatic interactions, hydrogen bonds, Van der Waals interaction, coordination bonds, and physical entanglement of polymers. Exemplary methods preparing the membrane include, but are not limited to, gelling methods like cross-linking alginate and similar polymers, molding, printing, and bonding.

In some embodiments, the gel-like material(s) is coated with one or more additional coating materials that prevent moisture/water evaporation and drying out. The additional coating material(s) may include, but are not limited to, shellac, poly(vinyl alcohol), polyvinylidene chloride, low density polyethylene, high density polyethylene, HPMC, gelatin, polyolefin, beeswax, lacquers, polypropylene, and ethylene vinyl alcohol. The additional coating material(s) may be coated on the gel-like material(s) in a single layer or multiple layers. The additional coating material(s) can be mixed with one or more other agents, including functional agents, wherein the other agents may include antimicrobial agents, plasticizers, film forming agents, hydrophobic agents, gel de-bonding agents, coloring agents, glitters, crosslinkers, hygroscopic agents, desiccants, and humectants.

In at least one aspect, the gel-like material is impregnated with a personal care product in a liquid solution, a suspension, an emulsion, or a powder form. For example, hand-soap may be added to a gelling mixture during a gel formation step to form a capsule that consists of or consists essentially of the gel-like material impregnated with soap. The soap may be released from the capsule upon application of sufficient force and/or in contact with water.

In at least one aspect, the gel-like material forms a membrane, which may be impregnated with a personal care product or without it, wherein the membrane encapsulates a personal care product in the core of the capsule, in a liquid solution, suspension, emulsion, solid or powder form. For example, body scrub is integrated into the gel-like membrane during gelling, and the core contains a body wash, such that upon application of force and/or water activation the capsules delivers both personal products; the user can decide to use both products, or to remove the scrub containing membrane and use only the body wash.

The disclosure also embodies a method of delivering a personal care product by packaging it in a gel-like material and releasing it under compression force squishing and breaking the capsule, and/or dissolving the capsule in contact with water. The gel-like capsule containing personal care product is flexible and pliable under pressure; it breaks open under minimal compression like finger- or palm-pressure. If the gel-like material contains water-soluble components, then the capsule will additionally dissolve in water releasing the personal care product. In the latter case the user can choose to apply both the compression and water activation or only one of the methods of release. The yield pressure is in the approximate order of a child's grip force. When the capsule is broken and or dissolved, the personal care product gets released into hands or onto skin, or body, and is then used in a conventional manner, e.g. rubbed on skin or body. If the capsule is not dissolvable, the membrane is collected upon the application of the product and is disposed.

In one application, the gel-like material encapsulating the personal care product is used to induce people to wash their hands or body more frequently and/or in a longer time. In one example, the gel-like material encapsulating a hand soap is used to induce children and adults to wash hands more frequently and/or in a longer time by endowing a fun texture and functionalities such as popping action, interesting shapes and colors.

In another application, the gel-like material encapsulating the personal care product is used to induce people to use a predetermined amount of the personal care product. In one example, the gel-like material encapsulating a hand soap contains the hand soap with the volume that is desirable for one hand wash such as 0.5 ml, 0.7 ml, 1 ml, 2 ml, or 3 ml. In another example, the gel-like material encapsulating a hand soap contains the hand soap with the volume that is lower than the desirable amount for one hand wash such as 0.05 ml, 0.1 ml, or 0.2 ml and the users can use two or more gel-like material encapsulating a hand soap to wash hands.

In other embodiments, the present disclosure provides capsules made of gel-like materials delivering single doses or servings of water, vitamin water, electrolytes, juices, alcohol, jello, pudding, jam, marmalade, spreads, chocolate, whipped cream, food, food additives, food colour, flavours, oil, vitamins, baking products, spices, candies, chewing gums, pet food, gasses and vapor, ice, etc. Similar to personal care products, these products can be integrated into the gel-like material or encapsulated by it. The gel-like material can be edible or non-edible and disposable.

Upon fabrication of the gel capsules, they can be dehydrated or lyophilized, and stored for longer shelf life or easier transportation, to be hydrated before use at a later time. They can also be frozen and defrosted before use at a later time.

Accordingly, in one aspect, there is provided a capsule containing personal care product comprising gel-like material containing one or more personal care products and uniformly filing the capsule, whereby the personal care product is provided in high concentration, whereby the personal care product includes but is not limited to personal hygiene products (hand soap, body wash, shampoo, conditioner, bubble bath, bath bombs cleanser, body and facial scrub, toothpaste, mouthwash, shaving cream, hair removal formulations, personal lubricant, hand sanitizer, anti-bacterial soap, disinfectant, deodorant, face wash, combination shampoo-conditioner-body wash, hair gel, mousse, nail polish remover, wax, pomade etc.), cosmetic products (moisturizer, detangler, cream, hand and body lotion, sunscreen lotion, lip balm, makeup, toner, additives, glitter, active ingredients, body paint, lip gloss, nail polish, hand cream etc.), and wellness products (vitamins, supplements, eye drops etc.) whereby the personal care product is in a liquid solution, suspension, emulsion, solid or powder form, whereby the gel-like material comprises but is not limited to water soluble, water semi-soluble, water insoluble components or any combination of aforementioned, including but not limited to locust bean gum polysaccharide, softening silicone, alginate, agarose, agar, pectin, xanthan, gelatin, carrageenan, arrowroot, cellulose derivatives, hyaluronan, elastin, chitosan, poly(vinyl alcohol), poly(acrylic acid), poly(ethylene glycol) and poly(vinyl acetate), whereby the gel-like materials have shapes including but not limited to spheres, ovals, rods, curved rods, stars, hemisphere, and other geometrical shapes, apples, lemons, bananas, donuts and other fruit and food shapes, bears, ducks, and other animal shapes, hands, feet, head, and other shapes of body parts, numbers or alphabets

In another aspect, there is provided a capsule containing personal care product comprising gel-like material forming a membrane surrounding a core filled with one or more personal care products, whereby gel-like material can also contain one or more personal care products that are same or different than the ones in the core, whereby the personal care product is provided in high concentration, whereby the personal care product includes but is not limited to personal hygiene products (hand soap, body wash, shampoo, conditioner, bubble bath, bath bombs cleanser, body and facial scrub, toothpaste, mouthwash, shaving cream, hair removal formulations, personal lubricant, hand sanitizer, anti-bacterial soap, disinfectant, deodorant, face wash, combination shampoo-conditioner-body wash, hair gel, mousse, nail polish remover, wax, pomade etc.), cosmetic products (moisturizer, detangler, cream, hand and body lotion, sunscreen lotion, lip balm, makeup, toner, additives, glitter, active ingredients, body paint, lip gloss, nail polish, hand cream etc.), and wellness products (vitamins, supplements, eye drops etc.), whereby the gel-like material comprises but is not limited to water soluble, water semi-soluble, water insoluble components or any combination of aforementioned, including but not limited to locust bean gum polysaccharide, softening silicone, alginate, agarose, agar, pectin, xanthan, gelatin, carrageenan, arrowroot, cellulose derivatives, hyaluronan, elastin, chitosan, poly(vinyl alcohol), poly(acrylic acid), poly(ethylene glycol) and poly(vinyl acetate), whereby the gel-like materials have shapes including but not limited to spheres, ovals, rods, curved rods, stars, hemisphere, and other geometrical shapes, apples, lemons, bananas, donuts and other fruit and food shapes, bears, ducks, and other animal shapes, hands, feet, head, and other shapes of body parts, numbers or alphabets.

In another aspect, there is a provided capsule containing personal care product comprising gel-like material and one or more functional coatings, whereby functional coatings prevent drying of the capsules, whereby functional coatings enhance stability of spheres, whereby functional coatings contain capsule dissolving agents.

There is also a provided method of delivery of personal care product comprising a capsule comprised of gel-like material and one or more personal care products, comprising, compressing the capsule between palms until it bursts whereby the compression force is in the approximate order of a child's grip force, applying the released personal care product on the body, removing left-over gel material from the body.

In another aspect, there is a provided method of delivery of personal care product comprising a capsule comprised of gel-like material and one or more personal care products, comprising: dissolving the capsule in water, applying the dissolved personal care product on the body.

There is also a provided method of fabrication of gel-like capsules containing personal care products comprising crosslinking of alginate and similar polymers, whereby drops of soap mixed with crosslinking agent are dropped into a solution of alginate for several seconds to several minutes to several hours, whereby the drops of soap mixed with crosslinking agent are frozen, whereby the drops of soap mixed with crosslinking agent are frozen in a mold that has whereby the gel-like materials have shapes including but not limited to spheres, ovals, rods, curved rods, stars, hemisphere, and other geometrical shapes, apples, lemons, bananas, donuts and other fruit and food shapes, bears, ducks, and other animal shapes, hands, feet, head, and other shapes of body parts, numbers or alphabets, whereby the fabricated gel-like capsules containing personal care products are washed with water to remove unreacted agents, whereby the fabricated gel-like capsules containing personal care products are exposed to one or more additional crosslinking agent(s) to alter the properties of the formed gel-like material, whereby the fabricated gel-like capsules containing personal care products are coated with one or more additional coating material(s), whereby the crosslinked alginate forms a flexible membrane, whereby the crosslinked alginate forms a gel crosslinked to the core.

In another aspect, there is also a provided method of fabrication of gel-like capsules containing personal care products comprising crosslinking of alginate and similar polymers, whereby drops of soap mixed with alginate are dropped into a solution of crosslinking agents for several seconds to several minutes to several hours, whereby the drops of soap mixed with alginate are frozen in a mold that has whereby the gel-like materials have shapes including but not limited to spheres, ovals, rods, curved rods, stars, hemisphere, and other geometrical shapes, apples, lemons, bananas, donuts and other fruit and food shapes, bears, ducks, and other animal shapes, hands, feet, head, and other shapes of body parts, numbers or alphabets, whereby the fabricated gel-like capsules containing personal care products are exposed to one or more additional crosslinking agent(s) to alter the properties of the formed gel-like material, whereby the fabricated gel-like capsules containing personal care products are coated with one or more additional coating material(s), whereby the crosslinked alginate forms a flexible membrane, whereby the crosslinked alginate forms a gel crosslinked to the core.

Further, for example, the present disclosure includes a composition comprising a gel-like material and a personal care product, wherein the composition is formulated to deform and burst upon application of a compression force less than 5 N or less than 3 N (the composition having a burst force less than 5 N or less than 3 N), and/or wherein the composition includes an outer membrane that comprises the gel-like material, the gel-like material comprising crosslinked alginate, and the outer membrane has a thickness in the range of about 0.1 mm to about 5 mm. For example, the composition may have a burst force in the range of about 0.5 N to about 2.5 N. In at least one example, the composition may comprise about 15% to about 99% water by weight, such as about 15% to about 20% water by weight, about 50% to about 99% water by weight, or about 60% to about 80% water by weight, with respect to the total weight of the composition. In some examples, the gel-like material may comprise a polysaccharide. Optionally, the polysaccharide may comprise locust bean gum polysaccharide, alginate, agarose, agar, pectin, xanthan, gelatin, carrageenan, arrowroot, cellulose derivatives, hyaluronan, chitosan, or a combination thereof. According to some aspects of the present disclosure, the personal care product may be a liquid or a gel. In at least one example, the personal care product may have a viscosity at 25° C. in the range of about 100 cps to about 4,000 cps. The personal care product may comprise, for example, hand soap, body wash, face wash, toner, shampoo, conditioner, a combination shampoo-conditioner-body wash, bubble bath, facial scrub, cleanser, body scrub, toothpaste, mouthwash, shaving cream, a hair removal formulation, a personal lubricant, hand sanitizer, anti-bacterial soap, disinfectant, deodorant, hair gel, mousse, wax, pomade, lotion, lip gloss, lip balm, nail polish, nail polish remover or hand cream. In at least one example, the gel-like material may be impregnated by the personal care product.

In some aspects, the composition may have a maximum cross-sectional dimension ranging from about 5 mm to about 15 mm, such as about 10 mm. In at least one example, the composition may have a spherical shape. Further, in some examples, the composition may comprise at least one coating (e.g., at least one coating material) that forms an outermost surface of the composition. For example, the at least one coating or coating material may comprise a de-bonding agent. In some examples, the at least one coating or coating material may include a first coating (or first coating material) and a second coating (or second coating material) disposed over the first coating (or first coating material), wherein the first and second coatings (or first and second coating materials) may comprise different materials. According to some aspects of the present disclosure, the composition may comprise an outer membrane comprising the gel-like material, and the personal care product may be enclosed within the outer membrane. For example, the outer membrane may be water insoluble. In at least one example, the outer membrane may comprise crosslinked alginate. In some examples, the outer membrane may have a thickness in the range of about 0.1 mm to about 3 mm. In other examples, the composition does not include an outer membrane or a coating. In some examples, an entirety of the composition is biodegradable.

Further, for example, the present disclosure includes a package comprising the composition immersed within an aqueous solution. In at least one example, the composition may be a gel-like capsule, and the package may contain a plurality of the gel-like capsules. In some examples, the aqueous solution may comprise about 95% to about 99% by weight water with respect to the total weight of the solution. Moreover, the present disclosure also is related to use of the composition, wherein the use may comprise applying compression force by a hand of a user to thereby release the personal care product. In some aspects, the compression force or burst force may be less than 5 N or less than 3 N, such as about 0.5 N to about 2.5 N. In some examples, the personal care product may comprise hand soap and/or the user may be a child.

The present disclosure also includes a composition comprising an outer membrane comprising a gel-like material, and a personal care product enclosed within (e.g., encapsulated within) the outer membrane. The personal care product may be a liquid or a gel, for example. The composition is formulated to deform and burst upon application of a compression force less than 5 N or less than 3 N (e.g., the composition having a burst force less than 5 N or less than 3 N). For example, the composition may have a burst force in the range of about 0.5 N to about 2.5 N. In some examples, the gel-like material may comprise a polysaccharide. For example, the gel-like may comprise locust bean gum polysaccharide, alginate, agarose, agar, pectin, xanthan, gelatin, carrageenan, arrowroot, cellulose derivatives, hyaluronan, chitosan, or a combination thereof. Further, for example, the personal care product may comprise hand soap, body wash, face wash, toner, shampoo, conditioner, a combination shampoo-conditioner-body wash, bubble bath, facial scrub, cleanser, body scrub, toothpaste, mouthwash, shaving cream, a hair removal formulation, a personal lubricant, hand sanitizer, anti-bacterial soap, disinfectant, deodorant, hair gel, mousse, wax, pomade, lotion, lip gloss, lip balm, nail polish, nail polish remover or hand cream.

In some aspects, the composition may comprise about 15% to about 99% water by weight, such as about 15% to about 20% water by weight, about 50% to about 99% water by weight, or about 60% to about 80% water by weight, with respect to the total weight of the composition. In some examples, the composition may have a burst force in the range of 0.5 N to 2.5 N. In some examples, the composition may be spherical. In at least one example, the composition may have a maximum cross-sectional dimension ranging from about 5 mm to about 15 mm. According to some aspects of the present disclosure, the outer membrane may have a thickness in the range of about 0.1 mm to about 5 mm, such as about 0.2 mm to about 3 mm.

The present disclosure also includes a composition comprising a personal care product, and a gel-like material impregnated with the personal care product, wherein the composition is formulated to deform and burst upon application of a compression force less than 5 N such as less than 3 N (e.g., the composition having a burst force less than 5 N or less than 3 N). In at least one example, the composition may further comprise at least one coating (e.g., at least one coating material) that forms an outermost surface of the composition. For example, the at least one coating may comprise a first coating (e.g., a first coating material) and a second coating (e.g., a second coating material), the first and second coatings comprising different materials. In some examples, the at least one coating or coating material may comprise a de-bonding agent. According to some aspects, the gel-like material may comprise locust bean gum polysaccharide, alginate, agarose, agar, pectin, xanthan, gelatin, carrageenan, arrowroot, cellulose derivatives, hyaluronan, chitosan, or a combination thereof. Further, for example, the personal care product may comprise hand soap, body wash, face wash, toner, shampoo, conditioner, a combination shampoo-conditioner-body wash, bubble bath, facial scrub, cleanser, body scrub, toothpaste, mouthwash, shaving cream, a hair removal formulation, a personal lubricant, hand sanitizer, anti-bacterial soap, disinfectant, deodorant, hair gel, mousse, wax, pomade, lotion, lip gloss, lip balm, nail polish, nail polish remover or hand cream.

The present disclosure also includes methods of applying a personal care product to a body of a user. For example, the method may comprise releasing the personal care product from a composition comprising a gel-like material and the personal care product, and applying the personal care product to the body of the user, wherein releasing the personal care product comprises applying a compression force by a hand of the user to deform and burst the composition. In some examples, the compression force is less than 5 N or less than 3 N. For example, the composition may have a burst force in the range of about 0.5 N to about 2.5 N. According to some aspects, the user is a child. In some examples, the personal care product may comprise a soap such as hand soap, and the gel-like material may comprise a polysaccharide. In at least one example, the gel-like material may comprise crosslinked alginate. Also included in the present disclosure, is a composition comprising an outer membrane comprising a gel-like material and a personal care product enclosed within the outer membrane, wherein the gel-like material comprises crosslinked alginate. For example, the outer membrane may have a thickness in the range of about 0.1 mm to about 5 mm.

### BRIEF DESCRIPTION OF DRAWINGS

FIGS. 1A-1C depict exemplary compositions according to the present disclosure in the shape of sphere (FIG. 1A), moon (FIG. 1B), and star (FIG. 1C).

FIGS. 2A-2D depict cross sections of exemplary compositions according to aspects of the present disclosure.

FIGS. 3A-3C depict process flow diagrams illustrating exemplary methods of delivering a personal care product via compositions of the present disclosure.

FIGS. 4A-4B illustrate exemplary methods of preparing compositions (e.g., personal care capsules) according to the present disclosure.

FIGS. 5A-5C depict schematics of exemplary equipment and processes for large scale automated manufacturing of compositions according to the present disclosure.

FIGS. 6A-6B depict additional shapes of exemplary compositions according to the present disclosure.

### DETAILED DESCRIPTION

The present disclosure generally relates to compositions formulated for delivery of personal care products. For example, the composition may be in the form of a capsule. The composition may comprise one or more gel-like materials. For example, the composition may comprise a flexible gel-like material and one or more personal care products integrated into the gel-like material or encapsulated within the gel-like material. The compositions herein may be formulated to deliver the personal care product upon the application of sufficient compression force, e.g., to deform and burst at least an outermost portion of the composition. The compression force may be relatively low to allow for bursting of the composition manually. For example, a person may exert a minimal burst force on a gel-like capsule of the present disclosure when holding it in their hand to dispense the personal care product. In some examples, the composition may be formulated to release the personal care product upon activation with water or an aqueous solution.

The terms “approximately” and “about” refer to being nearly the same as a referenced number or value. As used herein, the terms “approximately” and “about” generally should be understood to encompass ±10% of a specified amount or value.

Compositions according to the present disclosure may comprise a gel-like material or combination of gel-like materials and one or more personal care products. According to some aspects of the present disclosure, the composition may comprise an outer membrane that encloses one or more personal care products, wherein the outer membrane comprises one or more gel-like materials. Optionally, the outer membrane encloses a filling that consists of or consists essentially of the personal care product(s). Optionally, the outer membrane encloses a filling that consists of or consists essentially of one or more gel-like materials (which may be the same or different that the gel-like material(s) of the outer membrane) impregnated with one or more personal care products. According to some aspects of the present disclosure, the composition comprises one or more gel-like materials impregnated with one or more personal care products, with or without an outer membrane.

Exemplary personal care products suitable for the present disclosure include, but are not limited to, personal hygiene products (hand soap, anti-bacterial soap, shampoo, body wash, bubble bath, cleanser, body and facial scrub, conditioner, toothpaste, dental floss mouthwash, shaving cream, hair removal formulations, personal lubricant, hand sanitizer, disinfectant, deodorant, face wash, a combination shampoo-conditioner-body wash, hair gel, mousse, nail polish remover, wax, pomade etc.), cosmetic products (moisturizer, cream, lotion, sun lotion, lip balm, makeup, additives, glitter, active ingredients, body paint, lip gloss, nail polish, hand cream, etc.), and wellness products (vitamins, supplements, eye drops etc.). In some examples herein, the personal care product(s) comprises a soap or other type of cleaner such as, e.g., hand soap, anti-bacterial soap, body wash, face wash, or a combination shampoo-conditioner-body wash. The personal care product(s) may be in liquid, gel, or powder form. In the case of a liquid or gel (including e.g., soaps such as hand soap), the personal care product may have a viscosity at 25° C. in the range of about 2 cps to about 10,000 cps, such as about 100 cps to about 4,000 cps, for example. Viscosity may be measured by a viscometer, such as a rotary viscometer. Rotary viscometers measure viscosity by analyzing the torque required to rotate a spindle submerged in a fluid at a constant speed. A suitable viscometer may be a Vevor NDJ-8s Rotary Viscometer.

Exemplary gel-like materials include materials prepared from a gelling agent. Suitable gelling agents may comprise a divalent cation such as Ca²⁺, Sr²⁺, Ba²⁺, Pb²⁺, Cu²⁺, Cd²⁺, Co²⁺, Ni²⁺, Zn²⁺, Mn²⁺, or a combination thereof. In some examples, the gel-like material of the compositions herein comprises calcium (e.g., Ca²⁺), strontium (e.g., Sr²⁺), and/or barium (e.g., Ba²⁺). Exemplary gelling agents suitable for the present disclosure include, but are not limited to, calcium chloride, calcium lactate, calcium phosphate, barium chloride, strontium chloride, and combinations thereof. The choice of gelling agent is expected to have an effect on mechanical properties of the composition and/or the burst force (e.g., applied compression force) to release the personal care product from the gel-like material and from the composition. The gel-like material may comprise covalently crosslinked natural and/or synthetic polymers. Additionally or alternatively, the gel-like material may be biocompatible and/or biodegradable. Exemplary gel-like materials suitable for the present disclosure include, but are not limited to, alginate (including, e.g., calcium alginate, crosslinked alginate, dried sodium alginate film), carrageenan (including e.g., calcium carrageenan), poly(vinyl alcohol) (PVA) (including high molecular weight PVA), and gelatin.

According to some aspects of the present disclosure, the composition comprises one or more coating materials. Exemplary coating material(s) may include, but not limited to, shellac, poly(vinyl alcohol), polyvinylidene chloride, low density polyethylene, high density polyethylene, polyolefin, beeswax, lacquers, polypropylene, ethylene vinyl alcohol, or a combination thereof. The coating material(s) may be coated at least partially or completely on the gel-like material(s) in a single layer or multiple layers. The coating material(s) can be mixed with one or more other agents (which may be functional agents) such as antimicrobial agents, plasticizers, film forming agents, hydrophobic agents, gel de-bonding agents, coloring agents, glitters, crosslinkers, hygroscopic agents, desiccants, and/or humectants. The coating material(s) may provide additional functionalities including, but not limited to, altering the release rate of personal care products, drying protection, protection from microbial attack, enhancement of mechanical strength, coloring or modification of the surface appearance, modification of surface textures, facilitating the fabrication process, reducing syneresis, and/or surface sterilization. The coating material(s) may comprise, for example, one or more solubilizing agents, biocides such as formaldehyde, enzymes, organic solvents, surfactants, viscosity modifiers, film-forming agents, natural and synthetic gums, sequestering agents, starch, talc, fibers, and/or light screening chemicals.

The compositions herein (with or without an outer membrane, and/or with or without a coating material) may be formulated to deform and burst upon application of a relatively low compression force in order to release the personal care product(s) for use, e.g., for application to the body of a user. For example, the composition may be formulated to burse upon a compression force provided by the hand of a typical user. For example, the user may be a child or an adult, including elderly persons. According to some aspects of the present disclosure, the compression force may be less than 3 N (about 0.67 lbs), such as a compression force of about 0.5 N to about 2.5 N (about 0.11 lbs to about 0.56 lbs). As described further below, the compression force sufficient to burst the composition may be indicated by a burst force measured with a force gauge instrument.

The compositions herein may comprise at least 5% by weight, at least 20% by weight, or at least 50% by weight water with respect to the total weight of the composition. For example, the composition may comprise from about 5% to about 20% water by weight, such as about 5% to about 15%, from about 10% to about 20% or about 15% to about 20% by weight water, with respect to the total weight of the composition. Further, for example, the composition may comprise about 15% to about 99% water by weight, such as about 15% to about 20% water by weight, about 50% to about 99% water by weight, or about 60% to about 80% water by weight, with respect to the total weight of the composition

When the composition includes an outer membrane, the outer membrane may comprise a gel-like material as described herein, and/or the outer membrane may be water-insoluble, water soluble, or partially water soluble. Exemplary materials that may be used in a water-insoluble outer membrane include, but are not limited to, calcium alginate, calcium carrageenan, and covalently crosslinked natural and/or synthetic polymers. Exemplary materials that may be used in a water-soluble outer membrane include, but are not limited to, poly(vinyl alcohol) (PVA), dried sodium alginate film, and gelatin. Exemplary materials that may be used in a partially water soluble outer membrane include, but are not limited to, high molecular weight PVA that dissolves only in hot water, and mixtures of the foregoing materials used to form water-insoluble and water-soluble outer membranes (e.g., providing for a membrane with a water solubility between insoluble and readily soluble). The thickness of the outer membrane may be about 1 μm to about 10 mm, such as about 5 μm to about 1 mm, about 0.5 mm to about 1 mm, about 0.5 mm to about 1.5 mm, about 0.5 mm to about 1 mm, about 1 mm to about 2 mm, about 1.5 mm to about 2.5 mm, or about 2.4 mm to about 2.8 mm. In some examples where the composition is spherical, the thickness of the outer membrane may be about 5 mm or about 10 mm and may extend along most of the radius (e.g., 50% or more, or 75% or more of the radius), leaving a relatively small cavity for a personal care product.

Any of the compositions herein may be at least partially or completely biodegradable.

The compositions herein may be stored in a container, in water or an aqueous solution, e.g., to prevent the compositions from drying out. Such solutions may comprise at least 90% by weight water, e.g., about 95% to about 99% by weight water.

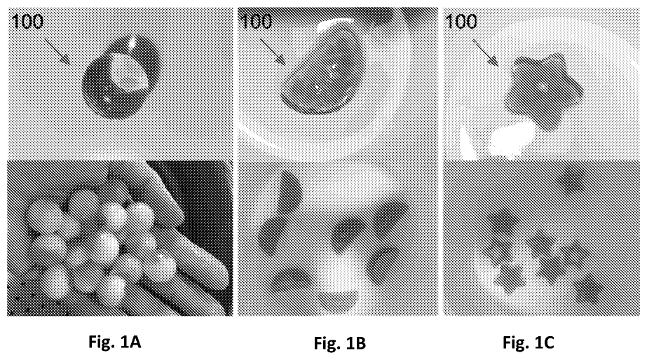

Exemplary compositions 100 are shown in FIGS. 1A-1C, depicting different shapes of the compositions. FIG. 1A illustrates the shape of a sphere, FIG. 1B illustrates the shape of a moon, and FIG. 1C illustrates the shape of a star.

The compositions herein may be in the form of gel-like capsules containing personal care products may comprise, consist of, or consist essentially of a gel-like material impregnated with a personal care product. In some examples, the composition may be in the form of a gel-like capsule comprising an outer membrane (e.g., a gel membrane) encapsulating one or more personal care products, wherein the personal care product(s) form a core of the composition contained within the outer membrane. In some examples, the composition may be in the form of a gel-like capsule having a hybrid structure, e.g., the composition comprising an outer membrane (e.g., a gel membrane) impregnated with one or more personal care products, the outer membrane encapsulating the same or different personal care product(s) in the core of the composition.

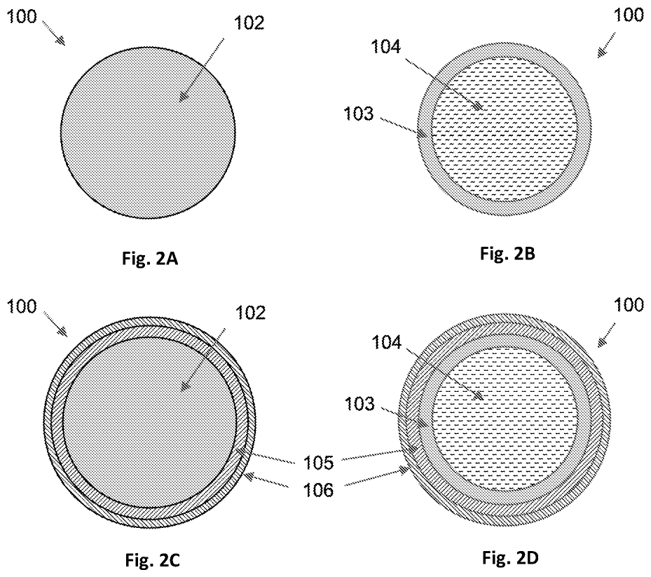

FIGS. 2A-D depict cross sections of exemplary compositions 100 according to the present disclosure. For example, FIG. 2A depicts a composition 100 comprising a personal care product integrated within a gel-like material 102 (e.g., the gel-like material being impregnated by the personal care product). FIG. 2B depicts a composition 100 comprising a core 104 that comprises a personal care product, the core 104 being encapsulated by an outer membrane 103 (e.g., a gel membrane or gel-like membrane). FIG. 2C depicts a composition 100 comprising a personal care product integrated with a gel-like material 103 (e.g., the gel-like material being impregnated by the personal care product), and a first coating material 105 (e.g., a first functional coating) and a second coating material 106 (e.g., a second functional coating). FIG. 2D depicts a composition 100 comprising a core 104 that comprises a personal care product, the core 104 being encapsulated by an outer membrane 103 (e.g., a gel membrane or gel-like membrane) and a first functional coating 105 and a second functional coating 106.

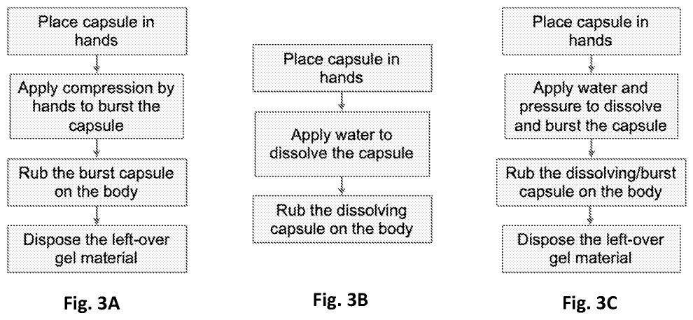

FIGS. 3A-C depict process flow diagrams illustrating exemplary methods of delivering a personal care product via compositions according to the present disclosure. FIG. 3A depicts delivery of an insoluble (e.g., water insoluble) composition (e.g., capsule). This exemplary method comprises steps of placing the composition in the hands of a user, applying compression by the hands to burst the composition, rubbing the burst composition on the body, and disposing of the left over gel material of the composition.

FIG. 3B depicts delivery of a soluble (e., water-soluble) composition (e.g., capsule). This exemplary method comprises steps of placing a composition (e.g., a capsule) in the hands of a user, applying water to dissolve the composition, and rubbing the dissolving capsule on the body.

FIG. 3C depicts delivery of a semi-soluble (e.g., at least partially soluble in water) composition (e.g., capsule). This exemplary method comprises steps of placing a composition (e.g., a capsule) in the hands of a user, applying water and pressure to dissolve and burst the composition, and disposing of the left-over gel material of the composition.

The compositions herein may have a maximum cross-sectional dimension ranging from about 0.5 mm to about 20 mm, such as about 1 mm to about 5 mm, about 1 mm to about 3 mm, about 5 mm to about 10 mm, about 5 mm to about 15 mm, about 7 mm to about 12 mm, about 10 mm to about 15 mm, about 12 mm to about 18 mm, or about 15 mm to about 20 mm. In the case of spherical compositions, the maximum cross-sectional dimension corresponds to the diameter. In the case of an egg-shaped or oval-shaped composition, the maximum cross-sectional dimension corresponds to the major axis. In the case of a rectangular or square-shaped composition, the maximum cross-sectional dimension corresponds to the inner diagonal distance. The composition size (e.g., cross-sectional size) may be measured using a suitable technique, such as a ruler, a digital caliper, etc.

As mentioned above, the compositions herein are formulated to allow for release of one or more personal care products encapsulated and/or integrated into the composition by the application of relatively low compression force. According to some aspects of the present disclosure, the compositions herein are formulated to deform and burst upon the application of a compression force less than 3 N (about 0.67 lbs), such as less than 2.5 N (about 0.56 lbs), or less than 2 N (about 0.45 lbs). For example, a compression force ranging from about 0.5 N to about 3 N may be sufficient to burst the composition. The composition may be sufficiently stable to withstand forces incidental to handling the composition, e.g., compression force less than 0.1 N (about 0.022 lbs).

The amount of force sufficient to burst the composition may be quantified by a force gauge to measure the burst force. The measurement may be made by placing the composition on a smooth flat, clean surface with the force gauge contact surface parallel and facing the smooth flat surface on which the composition is placed. The contact surface of the force gauge should be greater than the surface of the composition to provide for controlled consistent force being applied. The force gauge should be tared when held vertically prior to pressing down onto the composition. To measure burst force, the contact surface of the force gauge is advanced in a vertical direction towards the smooth flat surface to press on the composition with incremental force until the composition deforms and bursts. The movement of the force gauge contact surface should stop after the composition bursts, prior to touching the smooth bottom surface. The maximum or peak force number measured by the force gauge that is applied to the composition just before bursting is the burst force. An exemplary force gauge useful for measuring burst force is a Shimpo FGV-20XY digital force gauge, using a flat, circular surface (e.g., having a diameter of about 1 cm to about 3 cm, such as about 1.27 cm) without edges or sharp parts. The compositions herein may have a burst force less than 5 N, such as less than 3 N, e.g., less than 2.5 N, or less than 2 N. For example, the burst force may be in a range from about 0.5 N to about 5 N, about 0.5 N to about 3 N, about 1 N to about 4 N, about 1.5 N to about 3.5 N, or about 0.5 N to about 1.5 N, e.g., a burst force of about 0.7 N, about 1.0 N, about 1.2 N, about 1.5N, about 1.7 N, about 2.0 N, about 2.2 N. about 2.5 N, about 2.7 N, about 3.0 N, about 3.2 N, about 3.5 N, about 3.7 N, about 4.0 N, about 4.2 N, about 4.5 N, about 4.7 N, or about 5.0 N.

Exemplary Compositions with an Outer Membrane

The compositions may comprise a flexible gel-like capsule, wherein the capsule includes an outer membrane and one or more personal care products encapsulated by the membrane as a capsule core.

In a non-limiting example, the gel-like material forms a membrane, either impregnated with a personal care product or without it, that further encapsulates the same or a different personal care product in the core of the capsule, in a liquid solution, suspension, emulsion, solid or powder form. For example, body scrub powder may be integrated into the gel-like membrane during preparation of the composition via a gelling process, and the core contains a body wash, such that upon application of force and/or water activation the capsule delivers both personal products. In such cases, the user can decide to use both products, or to remove the scrub containing membrane and use only the body wash.

A flexible outer membrane according to the present disclosure may comprise, consist of, or consist essentially of a polymer cross-linked via chemical or physical interaction (e.g., electrostatic interaction, hydrogen bonding, or covalent bonding). The polymer may comprise, for example, alginate (e.g., sodium alginate), locust bean gum polysaccharide, softening silicone, agarose, agar, pectin, xanthan, gelatin, or a combination thereof. The membrane comprising one or more such polymers may include crosslinking, e.g., electrostatic interaction via calcium ions.

According to some aspects herein, the flexible outer membrane and personal care products encapsulated by the membrane are prepared by a gelation reaction between alginate (e.g., sodium alginate) and calcium (e.g., calcium chloride).

In some aspects of the present disclosure, the compositions herein and/or flexible gel-like material(s) thereof are packaged or otherwise stored in water or an aqueous solution, in a solution of the encapsulated personal care product(s), in a solution of one of more preservatives, in a solution of one of more antimicrobial agents, or in one or more oils. According to some aspects, the compositions herein and/or flexible gel-like material(s) thereof are packaged or otherwise stored in ambient conditions (e.g., at a temperature of about 25° C., a relative humidity of about 50%, and a pressure of about 1 atm.). According to some aspects, the compositions therein and/or flexible gel-like material(s) thereof are packaged or otherwise stored in a suitable environment, which may be a relatively dry environment (e.g., a relative humidity lower than 50%) or relatively a humid environment (e.g., a relative humidity greater than or equal to 50%) within a closed container. In at least one example, the composition may be packaged or otherwise stored in a relatively dry environment, e.g., in a closed container that does not contain liquid water, or an aqueous solution or other solution.

The compositions herein may comprise one or more functional coating materials. For example, according to some aspects of the present disclosure, the composition comprises a gel-like material (optionally in the form of an outer membrane) that is coated with one or more coating materials formulated to prevent moisture/water evaporation and drying out of the composition. For example, the composition may comprise only one or a plurality of coating materials (two or more coating materials, e.g., a first coating material and a second coating material). Exemplary coating material(s) may include, but not limited to, shellac, poly(vinyl alcohol), polyvinylidene chloride, low density polyethylene, high density polyethylene, polyolefin, beeswax, lacquers, polypropylene, ethylene vinyl alcohol, or a combination thereof. The coating material(s) may be coated at least partially or completely on the gel-like material(s) in a single layer or multiple layers. The coating material(s) can be mixed with one or more other agents (which may be functional agents) such as antimicrobial agents, plasticizers, film forming agents, hydrophobic agents, gel de-bonding agents, coloring agents, glitters, crosslinkers, hygroscopic agents, desiccants, and/or humectants.

According to some examples herein, the gel-like material(s) of the composition is integrated with one or more gel de-bonding agents. The gel de-bonding agents may be mixed with the gel-like material(s) when a user applies sufficient force to break a membrane of the composition including the gel-like material(s), and the gel-like material(s) partially or completely dissolves in water during or after use. The gel de-bonding agent(s) may be incorporated around the surface of the composition but separated from the gel-like material(s) via a physical barrier. Thus, for example, when the user creates a leak in the barrier, this may initiate mixing between the gel de-bonding agent(s) and the gel-like material(s). For a non-limiting example, a composition comprising a calcium alginate gel encapsulated with a personal care product may be coated with one or more coating materials mixed with calcium alginate gel de-bonding agent(s) that may include, for example, citric acid, ethylenediaminetetraacetic acid (EDTA), ethylene glycol-bis(β-aminoethyl ether)-N,N,N′,N′-tetraacetic acid (EGTA), alginate lysase, polyphosphate, or a combination thereof. In another non-limiting example, a composition comprising a calcium alginate gel encapsulated with a personal care product may be coated with one or more coating materials and one or more calcium alginate gel de-bonding agents are attached or otherwise coupled to at least a portion of the surface of the coating material(s).

In some exemplary uses according to the present disclosure, the composition comprises one or more gel-like materials encapsulating a personal care product, wherein the composition may be used to induce people to wash their hands or body more frequently and/or in a longer time/duration. In some examples, the composition comprises a gel-like material encapsulating a hand soap, wherein the composition may be used to induce children and adults to wash their hands more frequently and/or in a longer time/duration by endowing a fun texture and functionalities such as popping action, and/or interesting shapes and colors.

In some exemplary uses according to the present disclosure, the composition comprises one or more gel-like materials encapsulating a personal care product, wherein the composition may be used to induce people to use a predetermined amount of the personal care product, the predetermined amount being that included in the composition. In some examples, the composition comprises one or more gel-like material(s) encapsulating a hand soap, the volume of the hand soap being selected (e.g., desirable) for one hand wash. For example, the volume of hand soap in the composition may range from about 0.3 ml to about 5 ml, such as about 0.5 ml, about 0.7 ml, about 1 ml, about 2 ml, or about 3 ml. In some examples, the composition may be formulated for using two or more compositions simultaneously or consecutively. For example, the composition may comprise one or more gel-like materials encapsulating a hand soap, the volume of the hand soap being lower than a selected (e.g., desirable) amount for one hand wash. For example, the volume of hand soap in the composition may range from about 0.01 ml to about 0.5 ml, such as about 0.05 ml, about 0.1 ml, or about 0.2 ml. In such cases, the user can use two or more compositions to wash their hands.

Exemplary Compositions Impregnated Personal Care Product(s)

According to some aspects of the present disclosure, the composition does not include a membrane and a core. For example, the composition may comprise a flexible gel-like material that is impregnated with one or more personal care products. The personal care product(s) may be released out from the flexible gel-like material by a suitable method, such as breaking the gel-like material(s), diffusion, rubbing the gel-like material onto the delivery site (e.g., the hands and/or other portions of the body), or squeezing the gel-like material onto the delivery site (e.g., the hands and/or other portions of the body). The personal care product(s) can be distributed in the gel-like material homogeneously, multi-layered, in gradients, or in randomly distributed manner.

Hybrid Compositions

Additional compositions of the present disclosure may comprise combinations of the above examples, e.g., a composition comprising an outer membrane (e.g., gel membrane) impregnated with one or more personal care products, wherein the membrane encapsulates the same or a different personal care product in a core of the composition.

The following describes exemplary methods of preparing compositions according to the present disclosure. Using any of the methods below or combination of such methods, compositions can be fabricated providing an outer membrane impregnated with a personal care product encapsulating the same or different personal care product in the core of the composition. For example, sodium alginate solution may be mixed with a first personal care product (Mixture #1) and another, second personal care product may be mixed with calcium chloride (Mixture #2), wherein Mixture #2 is deposited into Mixture #1.

General Fabrication Methodologies

The following describes exemplary methods of preparing the compositions according to the present disclosure, e.g., methods 1, 2, 3, 4, and 5. It is understood that these methods are exemplary only and a person of ordinary skill in the art will recognize that aspects of each method may be combined with aspects of any other method.

Exemplary Method 1. Direct Spherification Using Sodium Alginate and Calcium Chloride Mixing

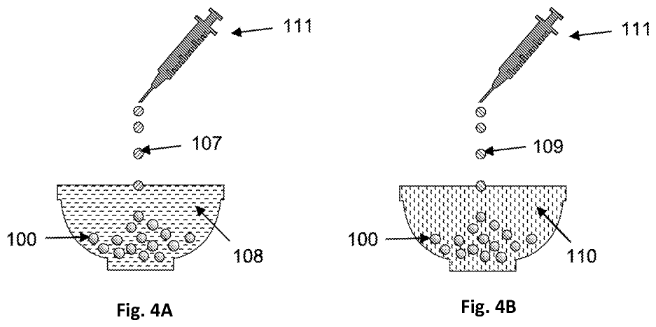

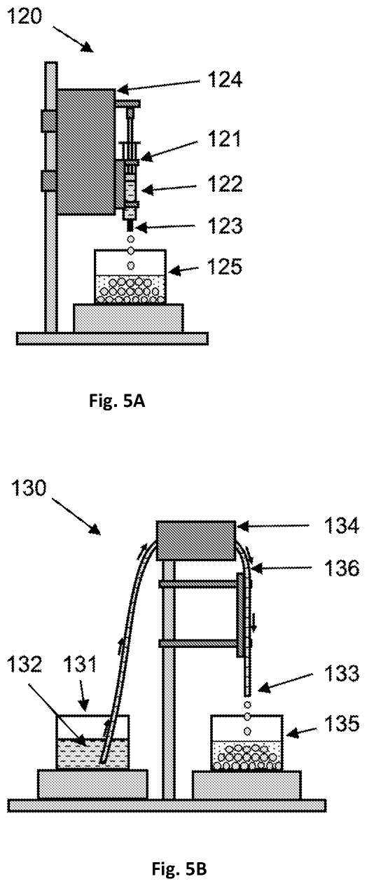

In an exemplary method according to the present disclosure, as summarized in FIG. 4A, a personal care product may be mixed with sodium alginate and discharged into a gelling agent solution in water. For example, sodium alginate powder may be dissolved in water in 0.1-10% w/v, e.g., 0.1-2% w/v. Optionally, a blender may be used to break down sodium alginate powder until no particles are observed (some loss of alginate may happen due to attachment of the particles on the blender wall). Optionally, sodium alginate may be dissolved in water using a colloid mill machine, which may eliminate the need to remove air bubbles that form as a result of dissolution. One or more personal care products may be intermixed into the sodium alginate solution with a volume ratio between 1:99 to 99:1, for example 5:5 to 9:1. Optionally one or more coloring agents can be added. In a separate container, a solution of one of more gelling agents in water may be prepared in concentrations ranging from 0.1% w/v to 10% w/v, such as 0.1% w/v to 2% w/v. The mixture of sodium alginate solution and personal care product may be discharged into the solution of gelling agent in a volume ranging from 10 μl to several milliliters. The discharged mixture may form a shell instantly or within several minutes generating a sphere or oval shape with a flexible outer membrane with the personal care product encapsulated in the core. After a predetermined time of reaction, the products (e.g., capsules) may be retrieved from the solution of gelling agent and placed into a washing bath to remove unreacted gelling agents and sodium alginate. This washing step can be repeated several times.

For example, FIG. 4A depicts a method of fabrication of personal care capsules 100 by dispensing from an injector 111 drops composed of soap and calcium 107 and gelling the drops composed of soap and calcium in an alginate solution 108.

In this exemplary Method 1, personal care products may include but are not limited to personal hygiene products (soap, shampoo, body wash, bubble bath, cleanser, body and facial scrub, conditioner, toothpaste, dental floss mouthwash, shaving cream, hair removal formulations, personal lubricant, hand sanitizer, disinfectant, deodorant, face wash, combination shampoo-conditioner-body wash, hair gel, mousse, nail polish remover, wax, pomade etc.), cosmetic products (moisturizer, cream, lotion, sun lotion, lip balm, makeup, additives, glitter, active ingredients, body paint, lip gloss, nail polish, hand cream etc.), wellness products (vitamins, supplements, eye drops etc.).

In Method 1, a gelling agent in the form of a compound containing a divalent cation such as Ca²⁺, Sr²⁺, Ba²⁺, Pb²⁺, Cu²⁺, Cd²⁺, Co²⁺, Ni²⁺, Zn²⁺, and/or Mn²⁺ may be selected, e.g., due to their low toxicity. The gelling agent(s) may include, but not limited to, calcium chloride, calcium lactate, calcium phosphate, barium chloride, and strontium chloride. It is believed that the gelling agent may at least partially affect the resulting mechanical properties of the gel-like material(s) of the composition, including, e.g., the burst force of the composition comprising the gel-like material encapsulating or encapsulated with the personal care product(s). Reaction of the alginate/personal care product/water mixture with the gelling agent solution may generally occur rapidly and produce a gel-like material promptly. Gelation generally proceeds from the outer surface to the center of the composition (e.g., in the form of a capsule, also referred to herein as a bead). Final gel properties such as hardness, strength, elasticity, size, durability, and burst force may be influenced by the time of residence in the gelling agent solution and may be optimized as desired given the disclosure herein and for the properties desired for each product. Generally, a residence time of 0.1-25 minutes may be used, but 1-10 minutes is usually sufficient.

It is believed that longer reaction time after discharging the mixture of sodium alginate and personal care products into the gelling agent solution in water may generate thicker outer membranes (also referred to herein as a shell) and eventually crosslinks the core to form a homogeneous solid.

The fabricated membrane may thicken gradually in 1 hour or in several days, provided that the unreacted alginate is present in the core and the divalent ions can reversibly migrate into the core.

One embodiment can have a flexible membrane on the surface of the beads encapsulated with personal care products. As mentioned above, the thickness of the flexible membrane can range from 1 μm to several millimeters. The ratio between the thickness and the maximum cross-sectional dimension (e.g., diameter in the case of a sphere) of the composition can range from 1:99 to 99:1. The users can apply pressure or force on the composition to break the membrane to release the personal care product(s).

In another embodiment, the composition encapsulating personal care products does not have core-shell structure and instead the core is filled with a solidified gel or gels. In such cases, the users can squeeze, rub, or break the compositions to release the encapsulated personal care products

To prevent dilution of the personal care products by mixing with sodium alginate solution in water, the sodium alginate can be directly dissolved into the personal care product to form a mixture of sodium alginate and personal care product.

The method of discharging the mixture of sodium alginate and personal care products may include but is not limited to drop-wise discharging from the outside of the gelling agent solution in water, direct injection into the gelling agent solution in water, jet injection, pressure-assisted injection, and discharging using multi-channel injectors.

The method of discharging the mixture of sodium alginate and personal care products can be an injection using a coaxial needle wherein the inner needle discharges the mixture of sodium alginate and personal care products and the outer needle discharges the gelling agent solution in water.

The mixture of sodium alginate and personal care products can be frozen and discharged into the gelling agent solution in water. The mixture of sodium alginate and personal care products can be frozen in a mold with a predetermined shape.

The compositions with flexible membrane encapsulating the personal care products can be dried to remove the water content partially or completely. The drying methods include and not limited to air-drying in ambient or higher temperature, freeze-drying, and vacuum drying. The dried compositions can be rehydrated to partially or completely regain the water content in the membrane polymer matrix and inside the flexible membrane. The volume of the compositions can be partially or completely restored after the rehydration.

The compositions with flexible membrane encapsulating the personal care products can be coated with one or more coating materials, e.g., for additional functionalities. Such functionalities may include, but are not limited to, altering the release rate of personal care products, drying protection, protection from microbial attack, enhancement of mechanical strength, coloring or modification of the surface appearance, modification of surface textures, facilitating the fabrication process, reducing syneresis, and surface sterilization. They include solubilizing agents, biocides such as formaldehyde, enzymes, organic solvents, surfactants, viscosity modifiers, film-forming agents, natural and synthetic gums, sequestering agents, starch, talc, fibers, and light screening chemicals. The additional coating agents include but are not limited to shellac, poly(vinyl alcohol) (PVA), poly(ethylene glycol) (PEG), beeswax, carrageenan, pectin, coloring agents, mica beads, titanium oxide particles, silicon oxide particles, glycine, propolis, gelatin, and polydimethylsiloxane (PDMS).

As mentioned above, the compositions herein can be stored in one or more of storage media including but not limited to water, a gelling agent solution in water, the personal care product used for the fabrication or a diluted solution of the personal care product used for the fabrication, and water-insoluble oil such as olive oil, mineral oil, and coconut oil.

The compositions with flexible membrane encapsulating the personal care products may be fabricated and placed into another gelling agent solution in water one or more times to modify crosslinking density of the flexible membrane.

The gel-like material may be manufactured using food processers, e.g., used for fabrication of popping boba.

Exemplary Method 2. Reverse Spherification

In another method, as shown in FIG. 4B, a personal care product may be mixed with gelling agents and discharged into sodium alginate solution in water. A gelling agent solution in water may be prepared in 0.1-10.0% w/v. One or more personal care product may be mixed with the gelling agent solution in water in a volume ratio between 1:99 to 99:1 and more preferentially 10:90 to 90:10. In a separate container sodium alginate powder may be dissolved in water in concentration ranging 0.1-10% w/v and more preferentially 0.1-2.0% w/v. Optionally, a blender may be used to break down sodium alginate powder, e.g., until few or no particles are observed. Optionally, sodium alginate may be dissolved in water, e.g., using a colloid mill machine, which may reduce or eliminate the presence of air bubbles that may form as a result of dissolution. The mixture of personal care product and gelling agent solution in water may be discharged in the sodium alginate solution in water in a volume of ranging from 10 μl to several milliliters. The discharged mixture may form a shell instantly or within several minutes generating a sphere or oval shape with a flexible outer membrane with the personal care product encapsulated in the core. It is believed that longer reaction time generates thicker membrane. After a predetermined time of reaction, the beads are retrieved from the solution of gelling agent and placed into a washing bath to remove unreacted gelling agents and sodium alginate. This washing step can be repeated several times.

For example, FIG. 4B depicts a method of fabrication of personal care capsules 100 by dispensing from an injector 111 drops composed of soap and alginate 109 and gelling the drops composed of soap and alginate in a calcium solution 110.

Personal care products include but are not limited to personal hygiene products (soap, shampoo, body wash, bubble bath, cleanser, body and facial scrub, conditioner, toothpaste, dental floss mouthwash, shaving cream, hair removal formulations, personal lubricant, hand sanitizer, disinfectant, deodorant, face wash, combination shampoo-conditioner-body wash, hair gel, mousse, nail polish remover, wax, pomade etc.), cosmetic products (moisturizer, cream, lotion, sun lotion, lip balm, makeup, additives, glitter, active ingredients, body paint, lip gloss, nail polish, hand cream, etc.), wellness products (vitamins, supplements, eye drops etc.).

A gelling agent may be used, e.g., a compound containing a divalent cation such as Ca²⁺, Sr²⁺, Ba²⁺, Pb²⁺, Cu²⁺, Cd²⁺, Co²⁺, Ni²⁺, Zn²⁺ and Mn²⁺, e.g. due to their low toxicity. The gelling agents include but not limited to calcium chloride, calcium lactate, calcium phosphate, barium chloride, and strontium chloride. Without being bound by theory, it is believed that the choice of gelling agent may have an effect on mechanical properties of the gel-like materials, and hence on the compositions, including for example the burst force of the composition, e.g., comprising the gel-like material encapsulated with the personal care products. Reaction of the alginate/personal care product/water mixture with the gelling agent solution may be relatively fast and may produce a gel-like material promptly. Gelation is generally initiated from the outer surface and proceeds outward. Final gel properties such as hardness, strength, elasticity, size, durability, and burst force may be influenced by the time of residence in the gelling agent solution and may be optimized for each product. Generally, a residence time of 0.1-25 minutes may be used, but 1-10 minutes is usually sufficient.

Without being bound by theory, it is believed that a longer reaction time after discharging the mixture of the gelling agent and personal care products into the sodium alginate solution in water generates thicker membrane shell with the thickness ranging from a few micrometers to several millimeters. During the reaction, the membrane shell grows outwards provided that the sodium alginate is present on the outside of the membrane and the gelling agent is present on the inside of the membrane that diffuse outwards. The embodiments adhering to a reverse spherification method do not crosslink inside the membrane shell provided the lack of sodium alginate in the core.

In some examples of the present disclosure, the composition comprises a flexible outer membrane encapsulated with one or more personal care products. The thickness of the flexible membrane can range from 1 μm to several millimeters. The ratio between the thickness and the diameter of the composition can range from 1:99 to 99:1. In this embodiment, the users can apply pressure or force on the composition to break the membrane to release the personal care product.

To prevent dilution of the personal care products by mixing with gelling agent in water, the gelling agent can be directly dissolved into the personal care product to form a mixture of the gelling agent and the personal care product.

The method of discharging the mixture of the gelling agent and the personal care products may include but is not limited to drop-wise discharging from the outside of the gelling agent solution in water, direct injection into the gelling agent solution in water, jet injection, pressure-assisted injection, and discharging using multi-channel injectors.

The method of discharging the mixture of the gelling agent and the personal care products can be an injection using a coaxial needle wherein the inner needle discharges the mixture of the gelling agent and the personal care products and the outer needle discharges the gelling agent solution in water.

The mixture of the gelling agent and the personal care products can be frozen and discharged into the gelling agent solution in water. The mixture of sodium alginate and personal care products can be frozen in a mold with a predetermined shape and size.

The compositions with flexible membrane encapsulating the personal care products can be dried to remove the water content partially or completely. The drying methods may include, but are not limited to, air-drying in ambient temperature (about 25° C.) or higher temperature, freeze-drying, and vacuum drying. The dried beads can be rehydrated to partially or completely regain the water content in the membrane polymer matrix and inside the flexible membrane. The volume of the compositions can be partially or completely restored after the rehydration.

The compositions with flexible membrane encapsulating the personal care products can be coated with one or more coating materials. Such coating materials may provide for additional functionalities including but not limited to altering the release rate of personal care products, drying protection, protection from microbial attack, enhancement of mechanical strength, coloring or modification of the surface appearance, modification of surface textures, facilitating the fabrication process, reducing syneresis, and surface sterilization. They include solubilizing agents, biocides such as formaldehyde, enzymes, organic solvents, surfactants, viscosity modifiers, film-forming agents, natural and synthetic gums, sequestering agents, starch, talc, fibers, and light screening chemicals. The additional coating agents include but are not limited to shellac, poly(vinyl alcohol) (PVA), poly(ethylene glycol) (PEG), beeswax, carrageenan, pectin, coloring agents, mica beads, titanium oxide particles, silicon oxide particles, glycine, propolis, gelatin, and polydimethylsiloxane (PDMS).

The compositions with flexible membrane encapsulating the personal care products can be stored in one or more of storage media including but not limited to water, a gelling agent solution in water, the personal care product used for the fabrication or a diluted solution of the personal care product used for the fabrication, and water-insoluble oil such as olive oil, mineral oil, and coconut oil.

The compositions with flexible membrane encapsulating the personal care products may be fabricated and placed into another gelling agent solution in water one or more times to modify crosslinking density of the flexible membrane.

Exemplary Method 3. Fabrication of Empty Shell and Soap Diffusion

In some aspects of the present disclosure, the composition may be initially prepared without a personal care product, wherein one or more personal care products may be incorporated into the composition later. For example, in an exemplary method, a composition comprising a flexible membrane may be fabricated without personal care products, and personal care products may be incorporated afterwards into the composition.

In a non-limiting example, sodium alginate may be dissolved in water in 0.1-10% w/v, e.g., 0.1-2% w/v. Optionally, one or more coloring agents can be added. In a separate container, a solution of one or more gelling agents in water may be prepared in concentrations ranging from 0.1% w/v to 10% w/v, such as 0.1% w/v to 2% w/v. The sodium alginate solution may be discharged into the solution of gelling agent in a volume ranging from 10 μl to several milliliter. The discharged mixture may form a shell instantly or within several minutes generating a sphere or oval shape with a flexible outer membrane with the core in liquid state. After a predetermined time of reaction, the products are retrieved from the gelling agent solution and placed into a washing bath to remove unreacted gelling agents and sodium alginate. This washing step can be repeated several times. The fabricated compositions with flexible membrane may be placed in a container with a personal care product allowing the personal care product to be transported inside the composition by diffusion in the time scale ranging from 1 hr to several days. The composition with flexible membrane encapsulating the personal care product may be retrieved and washed in water.

To fabricate the composition with flexible membrane without personal care products, alternatively the gelling agent solution in water can be discharged into the sodium alginate solution in water. A gelling agent solution in water may be prepared in 0.1-10.0% w/v. In a separate container, the sodium alginate may be dissolved in water in concentration ranging 0.1-10% w/v, such as 0.1-2.0% w/v. The gelling agent solution in water may be discharged in the sodium alginate solution in water in a volume of ranging from 10 μl to several milliliters. The discharged gelling agent solution in water may form a membrane (e.g., a shell) instantly or within several minutes generating a sphere or oval shape with a flexible outer membrane. Without being bound by theory, it is believed that longer reaction time generates thicker membrane. After a predetermined time of reaction, the compositions are retrieved from the solution of gelling agent and placed into a washing bath to remove unreacted gelling agents and sodium alginate. This washing step can be repeated several times. The fabricated compositions with flexible membrane may be placed in a container with a personal care product allowing the personal care product to be transported inside the bead by diffusion in the time scale ranging from 1 hr to several days. The compositions with flexible membrane encapsulating the personal care product(s) may be retrieved and washed in water.

Personal care products useful for this process may include, but are not limited to, personal hygiene products (soap, shampoo, body wash, bubble bath, cleanser, body and facial scrub, conditioner, toothpaste, dental floss mouthwash, shaving cream, hair removal formulations, personal lubricant, hand sanitizer, disinfectant, deodorant, face wash, combination shampoo-conditioner-body wash, hair gel, mousse, nail polish remover, wax, pomade etc.), cosmetic products (moisturizer, cream, lotion, sun lotion, lip balm, makeup, additives, glitter, active ingredients, body paint, lip gloss, nail polish, hand cream etc.), wellness products (vitamins, supplements, eye drops etc.).

A gelling agent may be used in the form of a compound containing a divalent cation such as Ca²⁺, Sr²⁺, Ba²⁺, Pb²⁺, Cu²⁺, Cd²⁺, Co²⁺, Ni²⁺, Zn²⁺ and Mn²⁺ due to their low toxicity. The gelling agents may include, but are not limited to, calcium chloride, calcium lactate, calcium phosphate, barium chloride, and strontium chloride. Without being bound by theory, it is believed that the choice of gelling agent may have an effect on mechanical properties of the gel-like material(s) and the composition, including the burst force of the composition comprising the gel-like material encapsulated with the personal care products. Reaction of the alginate/personal care product/water mixture with the gelling agent solution may be relatively rapid and may produce a gel-like material promptly. Gelation may be initiated from the outer surface and proceeds outward. Final gel properties such as hardness, strength, elasticity, size, durability, and burst force may be influenced by the time of residence in the gelling agent solution and may be selected based on the properties desired for each product. Generally, a residence time of 0.1-25 minutes may be used, but 1-10 minutes is usually sufficient.

In some aspects of the present disclosure, the composition may comprise an outer membrane that optionally comprises with one or more personal care products. The thickness of the flexible membrane can range from 1 μm to several millimeters. The ratio between the thickness and the diameter of the composition can range from 1:99 to 99:1. In this exemplary composition, a user can apply pressure or force on the composition to break the membrane to release the personal care product.

In some examples, the composition does not have a core-shell structure and instead the core may be filled with the solidified gels. In such cases, the user can squeeze, rub, or break the compositions to release the encapsulated personal care products.

The compositions with flexible membrane encapsulating the personal care products can be dried to remove the water content partially or completely. The drying methods include and are not limited to air-drying in ambient or higher temperature, freeze-drying, and vacuum drying. The dried compositions can be rehydrated to partially or completely regain the water content in the membrane polymer matrix and inside the flexible membrane. The volume of the compositions can be partially or completely restored after the rehydration.

The compositions with flexible membrane encapsulating the personal care products can be coated with one or more coating materials, e.g., for desired functionalities including but not limited to altering the release rate of personal care products, drying protection, protection from microbial attack, enhancement of mechanical strength, coloring or modification of the surface appearance, modification of surface textures, facilitating the fabrication process, reducing syneresis, and surface sterilization. They include solubilizing agents, biocides such as formaldehyde, enzymes, organic solvents, surfactants, viscosity modifiers, film-forming agents, natural and synthetic gums, sequestering agents, starch, talc, fibers, and light screening chemicals. The additional coating agents include but are not limited to shellac, poly(vinyl alcohol) (PVA), poly(ethylene glycol) (PEG), beeswax, carrageenan, pectin, coloring agents, mica beads, titanium oxide particles, silicon oxide particles, glycine, propolis, gelatin, and polydimethylsiloxane (PDMS).

The compositions with flexible membrane encapsulating the personal care products can be stored in one or more of storage media including but not limited to water, a gelling agent solution in water, the personal care product used for the fabrication or a diluted solution of the personal care product used for the fabrication, and water-insoluble oil such as olive oil, mineral oil, and coconut oil.

The compositions with flexible membrane encapsulating the personal care products may be fabricated and placed into a gelling agent solution in water one or more times to modify crosslinking density of the flexible membrane.

Exemplary Method 4. Empty Shell, Drying and Soap Diffusion Method

In another exemplary method according to the present disclosure, a composition comprising a flexible membrane may be fabricated without personal care products, dried partially or completely, and personal care products may be incorporated afterwards into the partially or completely dried bead with flexible membrane. Sodium alginate may be dissolved in water in 0.1-10% w/v and more preferentially 0.1-2% w/v. Optionally, one or more coloring agents can be added. In a separate container, a solution of one or more gelling agents in water may be prepared in concentrations ranging from 0.1% w/v to 10% w/v, such as 0.1% w/v to 2% w/v. The sodium alginate solution may be discharged into the solution of gelling agent in a volume ranging from 10 μl to several milliliter. The discharged mixture may form a shell instantly or within several minutes generating a sphere or oval shape with a flexible outer membrane with the core in liquid state. After a predetermined time of reaction, the composition products may be retrieved from the gelling agent solution and placed into a washing bath to remove unreacted gelling agents and sodium alginate. This washing step can be repeated several times. The fabricated compositions with flexible membrane may be placed in a container with a personal care product allowing the personal care product to be transported inside the composition by diffusion in the time scale ranging from 1 hr. to several days. The compositions with flexible membrane encapsulating the personal care product may be retrieved and washed in water.

Exemplary Method 5. Composition Dissolving

In another exemplary method of the present disclosure, the gel-like material may be partially or completely dissolved in water by incorporating gel de-bonding agents (e.g., the gel-bonding agents optionally being part of a coating material). For example, the gel de-bonding agents may be physically separated from the gel-like material. For example, the de-bonding agents may be applied to the gel-like material to unclog the drain. Further, for example, the gel-like material may be integrated with one or more gel de-bonding agents mixed with the gel-like material when the user breaks the membrane of the gel-like material, and the gel-like material may partially or completely dissolve in water during or after use. The gel de-bonding agents may be incorporated around the surface but separated from the gel-like material using a physical barrier, and when the user creates a leak in the barrier to initiate the mixing between the gel de-bonding agents and the gel-like material. For example, a calcium alginate gel encapsulated with personal care product may be coated with one or more additional coating material that is mixed with calcium alginate gel de-bonding agents including but not limited to citric acid, ethylenediaminetetraacetic acid (EDTA), ethylene glycol-bis(β-aminoethyl ether)-N,N,N′,N′-tetraacetic acid (EGTA), alginate lysase, and polyphosphate. In another example, the calcium alginate gel encapsulated with personal care product may be coated with one or more additional coating material and one or more calcium alginate gel de-bonding agents are attached on the surface of the coating material.

Exemplary Method 6. Large Scale Automated Manufacturing

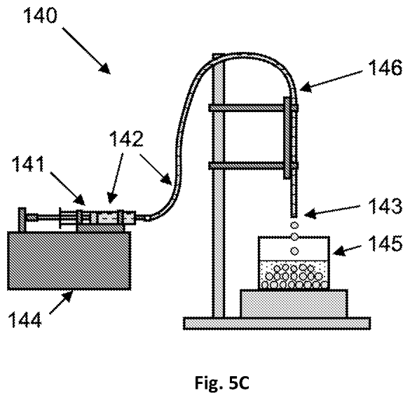

In another exemplary aspect of the present disclosure, the composition may be prepared by mixing a personal care product and gelling agent and adding the mixture in an injector directly assembled together with an injector pump and a reservoir and discharged into gelling bath. For example, FIG. 5A depicts a schematic of equipment 120 and processes for large scale automated manufacturing, where the mixture of personal care product and gelling agent 122 is added in an injector directly assembled together with an injector pump 124 and a reservoir 121, and discharged through injector tip 123 into gelling bath 125.

In an exemplary method, the mixture of personal care product and gelling agent may be added in a reservoir assembled together with an injector pump but separated from injector tip and discharged into gelling bath. For example, FIG. 5B depicts a schematic of equipment 130 and processes for large scale automated manufacturing, where the mixture of personal care product and gelling agent 132 is added in a reservoir 131 assembled together with an injector pump 134 but separated from injector tip 133 via connector 136, and discharged into gelling bath 135.

In some aspects of the present disclosure, the mixture of personal care product and gelling agent may be added in a reservoir separated from injector pump or injector tip and discharged into gelling bath. For example, FIG. 5C depicts a schematic of equipment 140 and processes for large scale automated manufacturing, where the mixture of personal care product and gelling agent 142 is added in a reservoir 141 separated from injector pump 144 or injector tip 143 via connector 146, and discharged into gelling bath 145.

The following examples are intended to illustrate the present disclosure without, however, being limiting in nature. It is understood that the present disclosure encompasses additional embodiments consistent with the foregoing description and following examples.

### EXAMPLES

### Example 1

Five grams of sodium alginate was added in 250 ml water by sprinkling the sodium alginate powder with simultaneous gentle mix with a spoon. The white chunks formed in the water were mixed vigorously and homogenized until no white powders are observed. The alginate solution was settled in ambient condition until all the air bubbles are released (from 1 to 24 hours, depending on the size of the chunks). A volume of 500 mL of liquid hand soap was poured in the alginate solution and gently mixed in a manner minimize the formation of foams or bubbles. The solution was homogeneously mixed. Dyes were added (e.g., food colors). The alginate/soap mixture was placed in a syringe and discharged dropwise into the gelling bath containing 5 g calcium chloride in 1L water. The droplets immediately formed a gel-like flexible coating encapsulating the liquid hand soap. After 5 seconds to a few minutes, the gel-like flexible coating encapsulating the liquid hand soap were collected using a perforated spoon or colander and washed in stopping bath to stop the gelling.

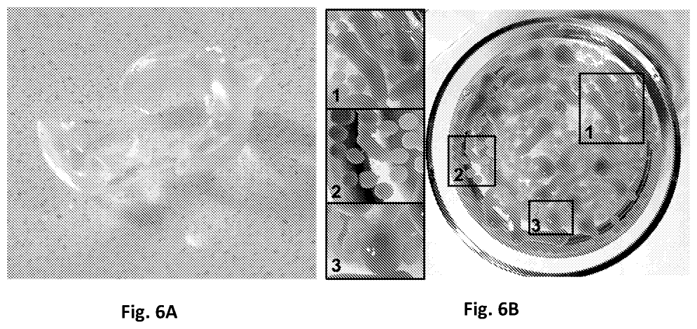

The size of the gel-like beads with flexible membrane encapsulating the liquid hand soap was ranging from 0.1 cm to lcm or 2 cm or 3 cm depending on the size of the droplet generated from the syringe. For the droplets with the average size of 0.5 cm, the reaction time of less than 5 minutes generated gel-like flexible membrane, and the reaction time of more than 5 minutes generated solid gel-like materials in the core as well. FIG. 6A depicts a capsule fabricated in a spherical shape with the reaction time of less than 5 minutes resulting in approximate size of 0.5 cm.

After the washing step, the gel-like beads maintained the flexible membrane up to a few days, and after a few days, the core was crosslinked to form gel-like solid beads.

### Example 2

This protocol was designed to prolong the presence time for the gel-like flexible membrane without crosslinking inside as described in Example 1. In Example 1, the mixture of sodium alginate and liquid soap was added dropwise into the gelling bath containing calcium chloride which quickly crosslinks the alginate to form a shell comprising calcium-alginate gel and a liquid core comprising soap and remaining uncrosslinked alginate molecules. However, even after thorough washing, calcium ions in the shell can slowly diffuse into the core which crosslinks the remaining alginate molecules. As a result, the core of the droplet slowly gels and the inner core crosslinks within a few hours or a few days to form a solid gel. In this Example 2, instead of having soap/alginate mixture added to calcium chloride gelling bath, soap was mixed with calcium chloride to form soap/calcium chloride solution, and this solution was added into sodium alginate solution. The core did not have uncrosslinked alginate, so the core remained liquid for a longer period of time.

In particular, five grams of sodium alginate was added in 250 ml water by sprinkling the sodium alginate powder with simultaneous gentle mix with a spoon. The white chunks formed in the water were mixed vigorously and homogenized until no white powders are observed. The alginate solution was settled in ambient conditions until all the air bubbles were released (from 1 to 24 hours, depending on the size of the chunks). In a separate container, 5 g calcium chloride was directly mixed into 500 ml of liquid hand soap without adding water. The soap/calcium chloride solution was drawn into a syringe. The injector tip was dipped into the sodium alginate solution, and the soap/calcium chloride solution was injected until the desired size is reached and retracted from the sodium alginate solution. The droplets immediately formed a gel-like flexible coating encapsulating the liquid hand soap. After 5 seconds to a few hours, the gel-like flexible coating encapsulating the liquid hand soap were collected using a perforated spoon or colander and washed in stopping bath to stop the gelling reaction.

The size of the gel-like beads with flexible membrane encapsulating the liquid hand soap was ranging from 0.1 cm to 1 cm or 2 cm or 3 cm or 4 cm or larger depending on the size of the droplet generated from the syringe. In addition, if the tip of the injector was dipped into the alginate solution to discharge the soap/calcium chloride solution, shapes including but not limited to spheres, ovals, rods, curved rods, donuts, numbers or alphabets could be formed by moving the injector or the bath during the injection. FIG. 6B depicts the shapes of rods or curved rods (1), spheres (2), and ovals (3) that were obtained by moving the injector or the bath during the injection process.

After the washing steps, the gel-like beads maintained the flexible membrane for several months (i.e. the extent of the test) and potentially longer.

### Example 3

Five grams of sodium alginate was added in 250 ml water by sprinkling the sodium alginate powder with simultaneous gentle mix with a spoon. The white chunks formed in the water were mixed vigorously and homogenized until no white powders are observed. The alginate solution was settled in ambient conditions until all the air bubbles are released (few hours). In a separate container, 5 g of calcium chloride was added into 500 ml water and mix thoroughly until all the calcium chloride was dissolved in water to form transparent solution. Liquid hand soap was then added in the calcium chloride solution and gently mixed until homogenous. The soap/calcium chloride solution was filled into a mold with a desired shape and placed in a freezer. The frozen soap/calcium chloride solution was discharged into the sodium alginate solution. A flexible membrane was formed immediately once the frozen soap/calcium chloride solution started to melt from the surface.

The size of the gel-like beads with flexible membrane encapsulating the liquid hand soap was ranging from 0.1 cm to 1 cm or 2 cm or 3 cm, 4 cm, 10 cm or larger depending on the size of the frozen soap/calcium chloride solution. In addition, using molds with different shapes, shapes including but not limited to spheres, ovals, rods, curved rods, stars, hemisphere, and other geometrical shapes, apples, lemons, bananas, donuts and other fruit and food shapes, bears, ducks, and other animal shapes, hands, feet, head, and other shapes of body parts, numbers or alphabets can be formed.

After the washing step, the gel-like beads maintained the flexible membrane for several months (i.e. the extent of the test) and potentially longer.

### Example 4

To test if the flexible membrane can be further hardened or potentially solidified, an additional calcium chloride solution in water in the concentration ranging from 0.5 to 2% w/v was prepared, and the gel-like spherical beads fabricated using the method in Example 3 were added into the calcium chloride solutions. The size of the gel-like spherical beads were reduced from 1.3 cm to 1.2 cm for 0.5% w/v calcium chloride solution in water, and from 1.3 cm to 1.1 cm for 2% w/v calcium chloride solution in water. This indicates that the flexible membrane can be further crosslinked after the initial fabrication process.

When dry calcium chloride salt particles were directly sprinkled on the surface of the fabricated gel-like beads, they were quickly dehydrated, leaked the contents (i.e. water and liquid soap), and were shrunk down to small amorphous structures. This also indicates that the flexible membrane can be further crosslinked after the initial fabrication process.

### Example 5

To test the storage methods, three groups of the gel-like beads with flexible membrane fabricated using the method in Example 3 were placed in one of the three containers: closed container with water, closed container without water, or open container without water. The gel-like beads stored in a closed container with water and those stored in closed container without water remained the shape and size for 2 months (i.e. extent of the study). The gel-like beads stored in open container without additional water was dried and collapsed to form a dried membrane containing the soap ingredients. When the dried membrane was placed in water, the dried membrane was rehydrated to form a sphere that has the same size and shape with the original gel-like bead before drying.

### Example 6

Five different batches of compositions (formulated as gel-like capsules) according to the present disclosure were prepared and tested to measure various properties of the capsules.

Each batch was manufactured by preparing a solution of water, liquid soap, and a calcium gelling agent. For batch 2, a combination of two different calcium gelling agents were used. The total amount of calcium gelling agent for each batch was about 0.2-3% w/v. For each batch, the liquid soap and the water were combined to have a volume ratio of 50:50. For batches 3, 4, and 5, a thickener in an amount of less than 0.5% w/v was also added to the soap and gelling agent solution. Separately, alginate solutions were prepared to have an alginate concentration in a range of 0.4-0.6% w/v.

For each batch, the soap and gelling agent solution was injected into the alginate solution to produce gel-like capsules in the shape of a sphere or an egg. The capsules were then washed to remove unreacted gelling agent(s) and alginate. Additionally, batches 4 and 5 were post-treated with different concentrations of a de-bonding agent to form a coating on the capsules.

The capsules produced in each batch had a flexible outer membrane with a liquid soap encapsulated therein. The thickness of the outer membranes ranged from about 0.2 mm to about 3.0 mm. The cross-sectional size (diameter) of the capsules ranged from about 6 mm to about 10 mm depending on the volume of liquid soap; the average of five capsule measurements for each batch of 100-200 capsules is reported in Table 1 below.

TABLE 1

| Diameter (cm) | Batch #1 | Batch #2 | Batch #3 | Batch #4 | Batch #5 |
|---|---|---|---|---|---|
| Test 1 | 0.93 | 0.75 | 0.64 | 0.89 | 0.88 |
| Test 2 | 0.91 | 0.69 | 0.56 | 0.91 | 0.90 |
| Test 3 | 1.0 | 0.70 | 0.63 | 0.90 | 0.91 |
| Test 4 | 0.87 | 0.75 | 0.62 | 0.96 | 0.98 |
| Test 5 | 0.89 | 0.74 | 0.61 | 0.99 | 0.92 |
| Average | 0.92 ± 0.05 | 0.73 ± 0.03 | 0.61 ± 0.03 | 0.93 ± 0.04 | 0.92 ± 0.04 |

Burst Force Test

A burst force test was conducted to measure the maximum force to break the capsules. Five capsules from each batch were tested. Burst force was measured with a Shimpo FGV-20XY digital force gauge using a flat, circular tip having a diameter of 1.27 cm without edges or sharp parts. The force gauge was programmed to peak measurement mode, by pressing the “PEAK” button.

Briefly, testing was conducted by placing each capsule onto a clean table. The force gauge tip was pressed down onto the capsule until the capsule burst. The tip of the force gauge was used to compress the capsule without hitting the table surface. The maximum or peak force number shown on the force gauge was recorded as the burst force. The table was cleaned between each measurement.

Table 2 reports the results for each of the five capsules tested for each batch. In each case, the burst force was less than 0.54 lbs. The average burst force for each batch of capsules was no more than about 0.38 lbs. Less force was needed to burst the smaller sized capsules from batches 2 and 3.

TABLE 2

| Burst Force (lbs) | Batch #1 | Batch #2 | Batch #3 | Batch #4 | Batch #5 |
|---|---|---|---|---|---|
| Test 1 | 0.54 | 0.23 | 0.23 | 0.14 | 0.08 |
| Test 2 | 0.40 | 0.14 | 0.12 | 0.22 | 0.09 |
| Test 3 | 0.31 | 0.21 | 0.21 | 0.31 | 0.19 |
| Test 4 | 0.37 | 0.08 | 0.09 | 0.22 | 0.44 |
| Test 5 | 0.26 | 0.19 | 0.15 | 0.26 | 0.24 |
| Average | 0.38 ± 0.1 | 0.17 ± 0.06 | 0.16 ± 0.06 | 0.23 ± 0.06 | 0.21 ± 0.1 |

Splash Test

A ball splash test was conducted to measure the amount of splash the capsules generated upon bursting. Five gel-like capsules from each batch were tested.

Briefly, to conduct each test, a 1.5 ft.×2 ft. sheet of paper was placed on the floor and secured by taping the corners of the paper to the floor. A single capsule was dropped from a height of 5 feet at the center of the paper. After the drop, the paper was collected. The maximum linear distance from the point on the paper where the capsule first landed upon dropping to the point at which the splash ended was measured and recorded. A new sheet of 1.5 ft.×2 ft. paper was used each time. Results are reported in Table 3. The greater amount of splash measured for Batch #4 and Batch #5 may be at least partially due to the presence of a de-bonding agent.

TABLE 3

| Splash (cm) | Batch #1 | Batch #2 | Batch #3 | Batch #4 | Batch #5 |
|---|---|---|---|---|---|
| Test 1 | 7 | 15 | 4 | 28 | 16 |
| Test 2 | 2 | 6 | 3 | 40 | 18 |
| Test 3 | 5 | 7 | 3 | 35 | 16 |
| Test 4 | 5 | 14 | 3 | 40 | 31 |
| Test 5 | 6 | 12 | 2 | 57 | 15 |
| Average | 5 ± 1.9 | 10.8 ± 4.1 | 3 ± 0.7 | 40 ± 10.7 | 19.2 ± 6.7 |

Box Drop Test

A box drop test was conducted to measure the durability of the capsules under typical shipping conditions.

Briefly, forty capsules from each batch were placed in an 8 oz. cylindrical plastic jar and sealed. An empty 5″×5″×5″ box was filled halfway with packing peanuts. The jar was firmly inserted into the center of the box. The rest of the box was then filled with packing peanuts and sealed shut with packing tape.

The sealed box was dropped from a height of approximately 2 m for a total of 6 times, so that each time the box was dropped a different side of the box was facing down. After the six drops, the jar was removed from the box. Table 4 below reports the number of capsules out of the total that were broken.

TABLE 4

| | Box Drop: # | | | | |
|---|---|---|---|---|---|
| | broken capsules out of 40 | | | | |
| | (% broken) | | | | |
| | Batch #1 | Batch #2 | Batch #3 | Batch #4 | Batch #5 |
| Test 1 | 2 | 1 | 10 | 0 | 0 |
| | (5%) | (2.5%) | (25%) | (0%) | (0%) |
| Test 2 | 1 | 3 | 20 | 0 | 0 |
| | (2.5%) | (7.5%) | (50%) | (0%) | (0%) |
| Test 3 | 3 | 0 | 17 | 0 | 0 |
| | (7.5%) | (0%) | (42.5%) | (0%) | (0%) |
| Average | 2 ± 1 | 1.3 ± 1.5 | 15.6 ± 5.1 | 0 | 0 |
| | (5 ± 2.5%) | (3.3 ± 3.8%) | (39 ± 13%) | (0%) | (0%) |

### Example 7

The burst force of compositions (in the form of gel-like capsules) according to the present disclosure was compared with the burst force of the following 5 commercially available products: Gelo Hand Soap Refill Pods-Foam (“Gelo 1”), Gelo Hand Soap Refill Pods-Liquid Gel (“Gelo 2”), doTERRA Triease Softgels Essential Oil Seasonal Blend (“doTERRA”), Eve Lom Cleansing Oil Capsules Travel Pack (“Eve Lom”), and Tide PODS Free and Gentle Laundry Detergent (“Tide PODS”).

The Gelo 1, Gelo 2, and Tide PODS products are each water-soluble pouches that contain cleansing agents such as soap and detergent, respectively. The pouches are made of poly (vinyl alcohol). The pouches are rectangular or square in shape and approximately 40-50 mm in size.

The doTERRA product contains an outer shell made of carrageenan, modified starch, cellulose, water and plasticizing agents; and a liquid fill of essential oils. The size is approximately 17 mm.

The Eve Lom product has a vegetable-based, biodegradable outer casing and a facial cleansing oil disposed inside. The size is typically 1-7 mm.

The burst force was measured for 5 samples of each type of product as described in Example 6. Results are provided in Table 5 below.

TABLE 5

| Burst | | | | | | Present |
|---|---|---|---|---|---|---|
| Force | | | | Eve | Tide | disclosure, |
| (lbs) | Gelo l | Gelo 2 | doTERRA | Lom | PODS | Batch #4 |
| Test 1 | 10.47 | 8.98 | >20 | >20 | 15.94 | 0.14 |
| Test 2 | 11.23 | 9.92 | >20 | >20 | 13.93 | 0.22 |
| Test 3 | 9.89 | 7.11 | >20 | >20 | 14.21 | 0.31 |
| Test 4 | 12.11 | 10.98 | >20 | >20 | 18.23 | 0.22 |
| Test 5 | 10.98 | 9.28 | >20 | >20 | 17.22 | 0.26 |
| Average | 10.936 | 9.254 | N/A* | N/A* | 15.906 | 0.23 |

*Above detection limit of the force gauge

The compositions of the present disclosure (samples from Batch #4 of Example 6) exhibited a lower burst force than each of the comparison products.

It is intended that the specification and examples be considered as exemplary only, with a true scope and spirit of the present disclosure being indicated by the following claims.

1. A composition comprising:
a gel-like material; and
a personal care product;
wherein the composition is formulated to deform and burst upon application of a compression force less than 5 N or less than 3 N; and/or
wherein the composition includes an outer membrane that comprises the gel-like material, the gel-like material comprising crosslinked alginate, and the outer membrane has a thickness in the range of about 0.1 mm to about 5 mm or about 0.2 mm to about 3 mm.

2. The composition of claim 1, wherein the composition has a burst force in the range of about 0.5 N to about 2.5 N.

3. The composition of claim 1, wherein the composition comprises about 50% to about 99% water by weight, with respect to the total weight of the composition.

4. The composition of claim 1, wherein the gel-like material comprises a polysaccharide and wherein the polysaccharide comprises locust bean gum polysaccharide, alginate, agarose, agar, pectin, xanthan, gelatin, carrageenan, arrowroot, cellulose derivatives, hyaluronan, chitosan, or a combination thereof.

5. The composition of claim 1, wherein the personal care product is a liquid or a gel.

6. The composition of claim 1, wherein the personal care product has a viscosity at 25° C. in the range of about 100 cps to about 4,000 cps.

7. The composition of claim 1, wherein the personal care product comprises hand soap, body wash, face wash, toner, shampoo, conditioner, a combination shampoo-conditioner-body wash, bubble bath, facial scrub, cleanser, body scrub, toothpaste, mouthwash, shaving cream, a hair removal formulation, a personal lubricant, hand sanitizer, anti-bacterial soap, disinfectant, deodorant, hair gel, mousse, wax, pomade, lotion, lip gloss, lip balm, nail polish, nail polish remover or hand cream.

8. The composition of claim 1, wherein the gel-like material is impregnated by the personal care product.

9. The composition of claim 1, wherein the composition has a maximum cross-sectional dimension ranging from about 5 mm to about 15 mm.

10. (canceled)

11. The composition of claim 1, wherein the composition comprises at least one coating that forms an outermost surface of the composition.

12. The composition of claim 11, wherein the at least one coating comprises a de-bonding agent.

13. The composition of claim 11, wherein the at least one coating includes a first coating and a second coating disposed over the first coating, the first and second coatings comprises different materials.

14. The composition of claim 1, wherein the composition comprises an outer membrane comprising the gel-like material, and the personal care product is enclosed within the outer membrane.

15. The composition of claim 14, wherein the outer membrane is water insoluble.

16. (canceled)

17. The composition of claim 14, wherein the outer membrane has a thickness in the range of about 0.1 mm to about 3 mm.

18. The composition of claim 1,
wherein the composition does not include an outer membrane or a coating.

19-40. (canceled)

41. A method of applying a personal care product to a body of a user, the method comprising:
releasing the personal care product from a composition comprising:
a gel-like material; and
the personal care product; and
applying the personal care product to the body of the user;
wherein releasing the personal care product comprises applying a compression force of less than 3 N by a hand of the user to deform and burst the composition.

42. (canceled)

43. The method of claim 41, wherein the composition has a burst force in the range of about 0.5 N to about 2.5 N.

44. The method of claim 41, wherein the user is a child.

45. The method of claim 41, wherein the personal care product comprises hand soap and the gel-like material comprises a polysaccharide.

46-48. (canceled)
